(19) United States
(12) Patent Application Publication
(10) Pub. No.: US 2025/0176892 A1
(43) Pub. Date: Jun. 5, 2025

(54) NEURAL PROBE

(71) Applicants: NFORMARE INC., Seoul (KR); YONSEI UNIVERSITY UNIVERSITY-INDUSTRY FOUNDATION, Seoul (KR)

(72) Inventors: Heon-Jin CHOI, Seoul (KR); Jae-Suk SUNG, Seoul (KR); Young-Cheol CHAE, Seoul (KR)

(21) Appl. No.: 18/841,052

(22) PCT Filed: Jan. 27, 2023
(86) PCT No.: PCT/KR2023/001273

§ 371 Date: Feb. 14, 2025

(30) Foreign Application Priority Data
Feb. 25, 2022 (KR) 10-2022-0024971

(51) Int. Cl. A61B 5/293

(52) U.S. Cl. CPC A61B 5/293; A61B 2562/164

(57) ABSTRACT

The present invention is intended to alleviate the burden upon insertion into a living body and to facilitate the handling of a neural probe, and provides a neural probe including a plurality of electrode parts to be inserted into the human body, wherein the plurality of electrode parts each include a flexible substrate, electrodes disposed on one side of the flexible substrate, and an insulation layer covering the one surface of the flexible substrate, and wherein the plurality of electrode parts are stacked.

### TECHNICAL FIELD

The present invention relates to a neural probe including a stacked structure.

### BACKGROUND ART

A neural probe refers to a micro-electrode element for neural interface that can measure neural signals at the final stage of an electronic medicine product or stimulate nerves by allowing current to flow thereto. A neural probe includes a micro-electrode element connected to a circuit module or a circuit module operating in conjunction with a micro-electrode element. Using a neural probe has the effect of treating nerve-related problems in the short or long term.

As an example, in the past, in order to measure brain signals, metal electrodes are inserted into the corresponding parts of the brain and measure action potentials. However, the brains of vertebrates are so complex that they consist of about 100 billion neurons, so that in order to elucidate brain circuits, there is required a system for measuring neural signals from multiple parts of the brain at the same time. For this purpose, neural probes are used to measure brain signals.

An electrode array for measuring signals is integrated at the end of the body of a neural probe, the signals measured from electrodes are transmitted to the outside through a wire formed along the probe, and dozens of electrodes can be integrated into a single probe body. Accordingly, signals can be measured simultaneously from multiple nerves. The form of the signals output in this case may be electrical signals, optical signals, or the like, and there is no particular limitation on the form of the signals. In addition, the probe can be equipped with a drug injection channel, and thus, can serve as a medium for delivering a drug.

The body of a neural probe is mainly made using a silicon material. This is adopted in an MEMS-type neural probe 1 such as that shown in FIG. 1. The probe 1 includes an electrode part 10 configured to be inserted into a nerve or cell, and a connection part 20 connected to the electrode part 10 and provided with terminals for connecting a main body and the electrode part 10. The body formed on a substrate made of a silicon material has a bulk shape having a specific thickness. There is a problem in that the electrode part 10 is easily broken when inserted and maintained in the human body or when handled, and also has a lack of flexibility, which acts as a limiting factor in selecting an effective location in the human body. Therefore, improvements are required for this.

Meanwhile, in the manufacture of a neural probe, in order to secure flexibility, a flexible neural probe in which electrodes are formed on a base substrate is manufactured. However, in this case, due to the thin thickness of a film itself, a problem such as bending may occur during manufacture, and there is a problem in which the electrodes are damaged or it is difficult to secure long-term durability when the neural probe is inserted into the human body. Accordingly, a neural probe having a manufacturing method or structure for securing durability needs to be manufactured. Durability can be secured by increasing the thickness. However, in this case, like a neural probe made of silicon, problems arise in that flexibility is significantly decreased or there is a concern about tissue loss due to the need for a large amount of space in the human body.

Furthermore, when a conventional neural probe is made of silicone by using a MEMS process, the overall product thereof is rigid rather than flexible. In particular, it has a brittle characteristic, so that there is a problem in that the mechanical stability thereof is considerably poor. Additionally, when a neural probe is implemented in a flexible form, the overall product thereof has flexibility, so that it also has a poor strength characteristic, it may be difficult to handle the neural probe when the neural probe is inserted into the human body, and a problem arises in that some parts requiring rigidity cannot be implemented.

In addition, when the conventional neural probe is made of silicon by using a MEMS process, there is a risk of damage attributable to external force due to its brittle nature. Accordingly, in order to secure stability against external force, it needs to be individually packaged using an air cushion or a cushioning material. As a result, the cost required for the packaging is high, and the handling is also difficult.

Furthermore, in the case of conventional neural probes, multiple electrodes are formed in a two-dimensional form to have a structure that measures signals or applies stimulation. However, these neural probes have limitations in terms of the precision of measurement or stimulation when they are used to measure bio-signals or apply bio-stimulation by using electrodes. In the case of neural probes in the form of MEMS, electrodes may be formed in a three-dimensional form, but problems arise in that it is difficult to fabricate three-dimensional electrodes and structures connecting the individual electrodes may become considerably complicated.

Furthermore, when the conventional neural probe is made of silicon by using a MEMS process, there are problems in that it is difficult to prevent the risk of damage to the brittle probe attributable to external force when the neural probe is inserted into the human body and the depth of insertion needs to be adjusted using a structure such as a screw, which causes the complexity of the structure of the neural probe.

Additionally, when electrodes that measure bio-signals or apply stimulation are formed in a neural probe, it is considerably important to obtain desired appropriate low impedance. However, in order to minimize patient discomfort during insertion into the human body and suppress damage to tissues such as nerves or cells, the size of the probe needs to be considerably small. The area of electrodes formed in accordance with the size of the probe is also considerably small, so that there is a problem of high impedance. Accordingly, there has not been proposed the structure of a neural probe that can simultaneously implement the function of allowing the neural probe to have low impedance and the function of suppressing damage to a patient's nerves, longitudinal tissues, etc. In particular, the conventional silicon-based MEMS-type neural probe has electrodes formed on only one surface thereof, so that it has limitations on the pursuit of lowering impedance while suppressing damage to a patient's tissues.

Furthermore, in the case of the conventional neural probes, the size of each of the neural probes needs to be minimized in order to minimize damage to tissues such as nerves and cells when the neural probe is inserted into a living body. To this end, the size of electrodes formed on the neural probe also needs to be smaller, so that there is a problem in that the small-sized electrodes have a limitation in terms of a measurement or stimulation target living body range when bio-signals are measured or bio-stimulation is applied.

In addition, in the case of the conventional neural probes, all the drive modules of each of the neural probes are installed externally and electrically connected to the neural probe. Accordingly, the number of external drive modules increases, the system structure becomes complex, the weight increases, and the handling thereof is expected to be difficult.

### DISCLOSURE

### Technical Problem

The present invention has been conceived to overcome the problems of the conventional art described above, and an object of the present invention is to fabricate a neural probe to be flexible, for example, by using a film, thereby forming the neural probe to be thin, alleviating the burden when the neural probe is inserted into a living body, and making the handling of the neural probe convenient.

### Technical Solution

In order to accomplish the above-described object, the present invention provides the following neural probes:

In an embodiment, the present invention is intended to alleviate the burden upon insertion into a living body and to facilitate the handling of a neural probe, and provides a neural probe including a plurality of electrode parts to be inserted into the human body, wherein the plurality of electrode parts each include a flexible substrate, electrodes disposed on one side of the flexible substrate, and an insulation layer covering the one surface of the flexible substrate, and wherein the plurality of electrode parts are stacked.

In an embodiment, the present invention provides a neural probe including an electrode part configured to be inserted into a human body, and a connection part connected to the electrode part and provided with a terminal that is electrically connected to electrodes; wherein the electrode part and the connection part each include a flexible substrate, a wiring formed on the substrate, and an insulation layer covering the substrate and the wiring; wherein the electrode part includes the electrodes disposed on one surface of the substrate and connected to the wiring; wherein the connection part includes a terminal formed on one surface of the substrate and connected to the wiring; and wherein the connection part includes a dummy pattern portion separated from the electrodes or terminal.

In an embodiment, the connection part may include: a connection part base substrate; the terminal formed on one surface of the connection part base substrate; a wiring electrically connecting the terminal and the electrode part on the one surface of the connection part base substrate; and the dummy pattern portion formed on the connection part base substrate while being spaced apart from the terminal and the wiring.

In an embodiment, the insulation layer may include a first insulation layer and a second insulation layer having higher flexibility than the first insulation layer, part of the insulation layer covering the substrate may be the second insulation layer, and an insulation layer covering the connection part and an insulation layer covering a portion of the electrode part connected to the connection part may be the first insulation layer.

In an embodiment, a drive circuit connected to the electrodes may be mounted at a location corresponding to the first insulation layer.

Furthermore, in an embodiment, the neural probe may further include a protective film covering at least one surface of the electrode part.

In an embodiment, the electrodes may include a first electrode disposed on one surface of the substrate and a second electrode disposed on the other surface thereof, and the first electrode disposed on the one surface of the substrate and the second electrode disposed on the other surface thereof may be disposed at locations corresponding to each other.

In an embodiment, the neural probe may further include a via or through hole formed through the substrate to connect the first and second electrodes.

In an embodiment, a plurality of electrode parts may be connected to the connection part, a spacer layer may be disposed between the plurality of electrode parts, at least the spacer layer may include a first spacer portion made of a bio-insoluble adhesive and a second spacer portion made of a bio-soluble adhesive, the first and second spacer portions may be disposed in the spacer layer, and the second spacer portion may be disposed on an end side to be inserted into the human body.

### Advantageous Effects

According to the present invention described above, the present invention allows a neural probe to be fabricated to be flexible, for example, by using a film, thereby forming the neural probe to be thin, alleviating the burden when the neural probe is inserted into a living body, and making the handling of the neural probe convenient.

### DESCRIPTION OF DRAWINGS

FIG. 1 is a drawing showing a conventional MEMS-type neural probe;

FIGS. 2 and 3 are drawings showing the electrode structures of neural probes according to embodiments of the present invention;

FIG. 4 shows a partial sectional view and plan view of the electrode structure of a neural probe according to an embodiment of the present invention;

FIGS. 5 and 6 are drawings showing the simulation results of a case where there is no insulation layer in an electrode and a case where an electrode is covered with an insulation layer including a through hole;

FIG. 7 shows a partial sectional view and plan view of the electrode structure of a neural probe according to another embodiment of the present invention;

FIG. 8 is a drawing showing the simulation results of a case where one electrode is covered with an insulation layer including a plurality of through holes;

FIG. 9 is a schematic diagram of a neural probe according to an embodiment of the present invention;

FIG. 10 shows sectional views of the electrode parts of neural probes according to other embodiments of the present invention;

FIG. 11 shows a sectional view of the electrode part of a neural probe according to still another embodiment of the present invention;

FIG. 12 shows a perspective view of the electrode part of the neural probe of FIG. 11;

FIG. 13 shows a schematic view of a state in which the neural probe of FIG. 11 is inserted into the human body;

FIG. 14 is a schematic diagram of a neural probe according to another embodiment of the present invention;

FIG. 15 is a schematic diagram of a state in which the neural probe of FIG. 14 is inserted into the human body;

FIG. 16 is a schematic diagram of a neural probe of still another embodiment of the present invention;

FIG. 17 is a sectional diagram of the electrode part of a neural probe according to still another embodiment of the present invention;

FIG. 18 shows a sectional view of the electrode part of a neural probe of still another embodiment of the present invention;

FIG. 19 shows a schematic diagram of a state in which the neural probe of FIG. 18 is inserted into the human body;

FIG. 20 shows a schematic diagram of a state in which the neural probe of another embodiment is inserted into the human body; and

FIG. 21 is a schematic diagram of a neural probe of still another embodiment of the present invention.

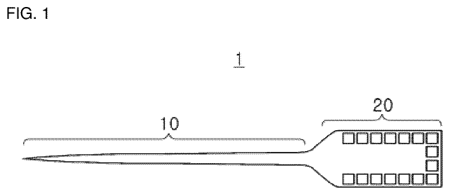

### DESCRIPTION OF REFERENCE SYMBOLS

1: neural probe 10: electrode part
11: base film 12: spacer layer
15: insulation layer 16: through hole
20: connection part 30: electrode
33: via hole 34: through hole
40: wiring 60: switching element
90: drive circuit

### MODE FOR INVENTION

Referring to the accompanying drawings, preferred embodiments will be described in detail below so that those having ordinary knowledge in the art to which the present invention pertains can easily practice the present invention. However, in the following detailed description of preferred embodiments of the present invention, when it is determined that a specific description of a related known function or configuration may unnecessarily obscure the gist of the present invention, the detailed description will be omitted. Furthermore, the same symbols are used throughout the drawings for the parts that perform similar functions and operations. Moreover, in the present specification, terms such as ‘upper,’ ‘upper part,’ ‘upper surface,’ ‘lower,’ ‘lower part,’ ‘lower surface,’ ‘side surface’, etc. are based on the drawings, and in reality, they may vary depending on the direction in which elements or components are disposed.

In addition, throughout the specification, when a part is described as being ‘connected’ to another part, this includes not only a case where they are ‘directly connected to each other,’ but also a case where they are ‘indirectly connected’ to each other with another element interposed therebetween. Furthermore, ‘including’ a component means that, unless otherwise specifically stated, another component is not excluded, but may be further included.

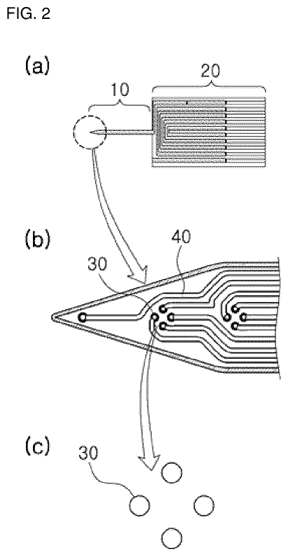

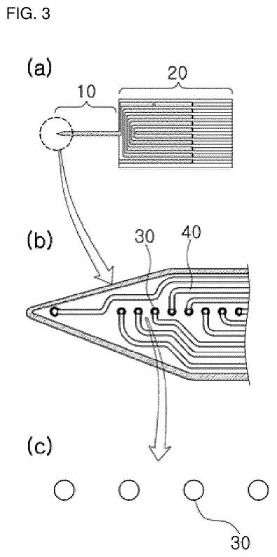

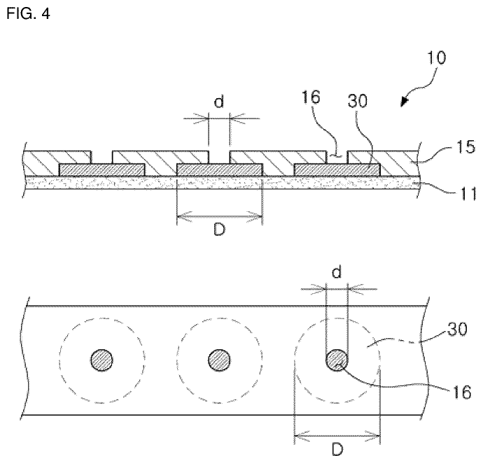

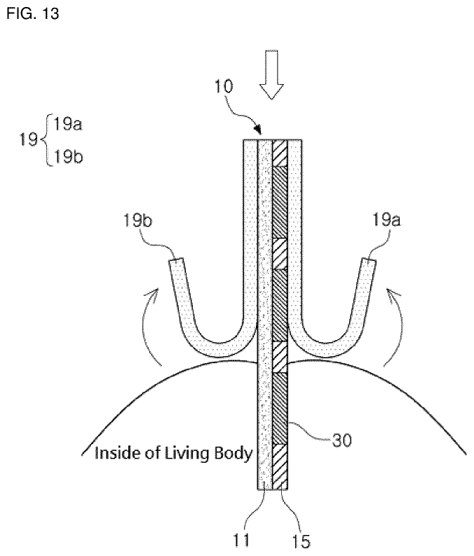

As shown in FIGS. 2 and 3, the electrode structure of a neural probe according to an embodiment of the present invention includes an electrode part 10 and a connection part 20, and the electrode part 10 and the connection part 20 are implemented by using a base film in the form of a film as a substrate. The electrode part 10 includes a base film 11 (see FIG. 4), electrodes 30 formed on the base film 11, and a wiring 40 connected to the electrodes 30 and connected to a measurement circuit 70 (see FIG. 13) or stimulation circuit 80 (see FIG. 13) configured in the outside of the electrode structure, the electrode part 10, or the connection part 20. The electrode part 10 may be inserted into a nerve or the like, and the electrode part 10 may be connected to the connection part 20 for connection to the main body of a separate electrode module or may be directly connected to the main body of the electrode module. In the case of the connection part 20, terminals may be included such that they can be connected to the main body of the electrode module by wire bonding or the like.

The electrodes 30 may be disposed in a tetrode-type arrangement as shown in FIG. 2 (c) or a linear-type arrangement as shown in FIG. 3 (c).

In the present invention, the electrode structure refers to the structure of the electrode part 10 configured to be inserted into the human body. When it is combined with a separate main body, it may include the connection part 20 for combining the electrode part 10 with the main body. When the main body is connected to the electrode part 10 in an integrated manner, only the electrode part 10 may be included as the electrode structure.

FIG. 4 shows a partial sectional view and plan view of the electrode structure of a neural probe according to an embodiment of the present invention.

The electrode part 10 of the electrode structure includes a base film 11; electrodes 30 formed on one surface of the base film 11; and an insulation layer 15 partially covering the base film 11 and the electrodes 30.

The base film 11 may be made of a polymer such as polyimide, and may have a thin thickness of 1 mm or less. However, the material or thickness thereof may be changed according to the required conditions. The electrodes 30 and the insulation layer 15 may be formed by applying a semiconductor process to the base film 11.

The base film 11 is fabricated to be flexible by using a base film 11 made of a flexible material, thereby forming the neural probe to be thin, alleviating the burden when the neural probe is inserted into a living body, and making the handling of the neural probe convenient.

The electrodes 30 are formed on the base film 11. Although not shown, the wiring 40 connected to the electrodes 30 is also formed. The electrodes 30 are formed in such a manner that a conductive material is applied onto the base film 11 by coating, deposition or plating, but the manufacturing method is not limited thereto and various methods may be applied.

The electrodes 30 may have lower impedance when formed to be larger. However, as the size of the electrodes increases, it becomes more difficult to specify a bio-signal measurement target nerve or cell, making it difficult to identify the problem of a specific nerve or cell. In the present embodiment, the electrodes 30 are covered with the insulation layer 15, through holes 16 are formed in the insulation layer 15 to expose parts of the electrodes 30 to the outside, and the electrodes 30 measure or stimulate a nerve or cell through the surfaces exposed as described above, i.e., the exposed surfaces.

The through holes 16 are formed by locally exposing only electrode portions requiring exposed after the application or stacking of the insulation layer 15 by using a method such as laser, lithography, or etching. The formation of the through holes 16 may easily control the size and depth of the electrodes, exposed by the through holes 16, by controlling the focus and intensity of the laser, controlling the photo intensity of lithography, or controlling the concentration of etchant.

The area of the electrodes 30 is larger than the area of the through holes 16, and accordingly, parts of the surfaces of the electrodes 30 are covered with the through holes 16. In the case where the electrodes 30 are circular, the diameter D of the electrodes 30 is larger than the diameter d of the through holes 16. The planar shape of the electrodes 30 is not limited to a circular shape. It is obvious that the electrodes 30 may have various shapes such as a square, a polygon, and an ellipse. It may be preferable that the area of the through holes 16 is 90% or less of the area of the electrodes 30, but the size may be changed depending on the shape of the electrode part 10.

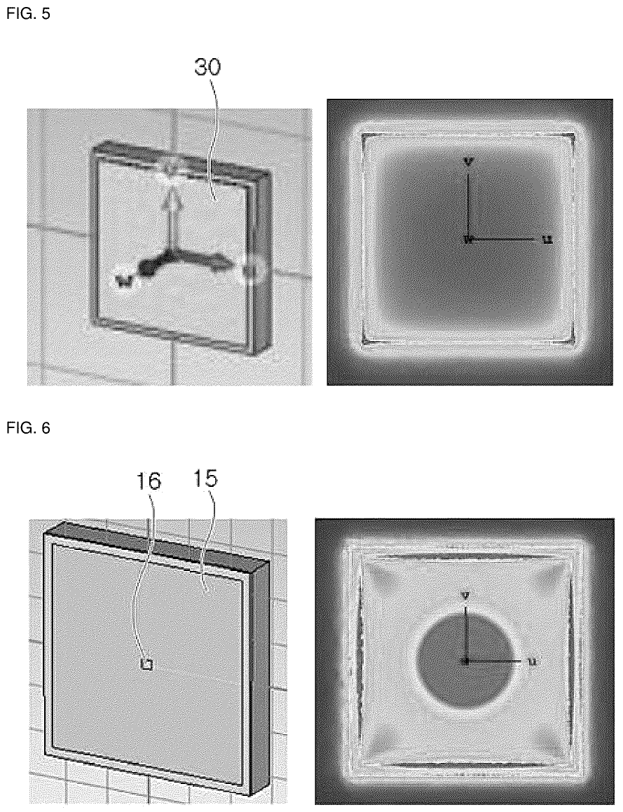

FIGS. 5 and 6 show the results of simulating the current density according to the through holes 16. In the case of FIG. 5, an electrode 30 is not covered with an insulation layer 15, and in FIG. 6, an electrode 30 is covered with an insulation layer 15 and then a through hole 16 is formed. The area of the through hole 16 is approximately 4% of the area of the electrode 30 of FIG. 5. When the experiment was performed under the same conditions, it was found that the current density was high around the through hole 16, as shown in FIG. 6. Compared to that of FIG. 5, a current density approximately 80 times higher than the current density of FIG. 5 was found in the through hole 16 of FIG. 6. Accordingly, accurate stimulation or measurement may be performed at an accurate location.

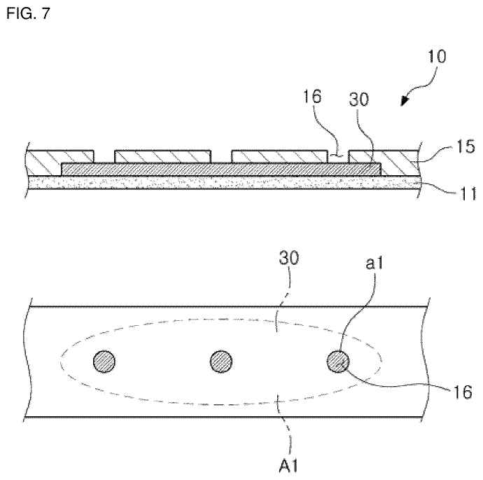

FIG. 7 shows a partial sectional view and plan view of the electrode structure of a neural probe according to another embodiment of the present invention.

The electrode part 10 of the electrode structure includes a base film 11; an electrode 30 formed on one surface of the base film 11; and an insulation layer 15 partially covering the base film 11 and the electrode 30.

Basically, the present embodiment is the same as the embodiment of FIG. 4 except for the electrode 30 and the through holes 16, so that the following description will be given with a focus on the differences therebetween.

The electrode 30 formed on the base film 11 is covered with the insulation layer 15, and the insulation layer 15 includes a plurality of through holes 16 at locations corresponding to the electrode 30. Accordingly, the area A1 of the electrode 30 is larger than the combined area al of the corresponding plurality of through holes 16.

This structure may also increase the current density compared to the case where the overall area of the electrode 30 is exposed without the through holes 16. Accordingly, accurate stimulation or measurement may be performed at an accurate location.

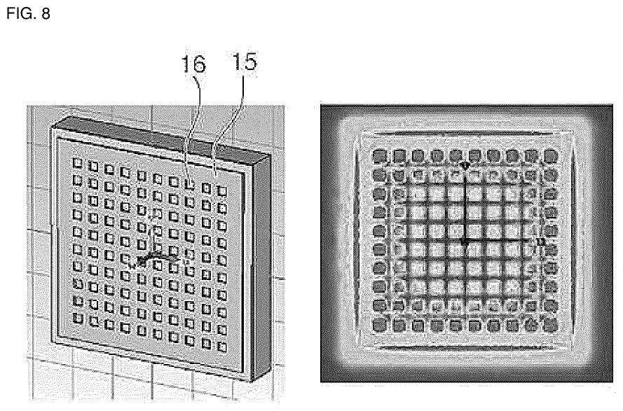

FIG. 8 shows the results of simulating the current density when a plurality of through holes 16 are formed in one electrode. The simulation was performed under the same conditions as the simulation of FIGS. 5 and 6 above. In FIG. 8, the plurality of through holes 16 are formed in the insulation layer 15, and the area of the through holes 16 is approximately 20% of the area of the electrode 30. In this case, the average current density was increased about twice compared to that in the case where the electrode 30 was not covered with the insulation layer 15. Accordingly, even when the plurality of through holes 16 are formed, accurate measurement and stimulation may be performed, and stimulation and bio-signal measurement functions may be implemented in a single neural probe by improving the electrode structure of the neural probe.

As described above, in order to lower the impedance of the electrode 30 in an embodiment of the present invention, the size of the electrode 30 may be configured to be as large as possible, and only an exposed area may be configured to be small. Since the impedance of the electrode decreases as the contact area of the electrode increases, it is assumed that the impedance is lowered by increasing the size of the electrode, and thus, an environment in which high energy can be transmitted is constructed. In particular, when only an exposed area is configured to be small, high energy may be concentrated, generated and transmitted from an electrode having low impedance.

The sectional view of the electrode probe coated with the electrical insulation layer so that the size of the exposed area is smaller than the size of the electrode(s) is shown for each of the embodiments of FIG. 4 and FIG. 7. When only part of the electrode is exposed in this manner, the stimulation energy of the electrode for bio-stimulation is particularly concentrated on the exposed area, so that energy having a high stimulation value is intensively transmitted to a local nerve or cell, thereby performing effective stimulation. Furthermore, a plurality of nerves or cells may be stimulated with high energy at the same time by providing a plurality of exposed areas.

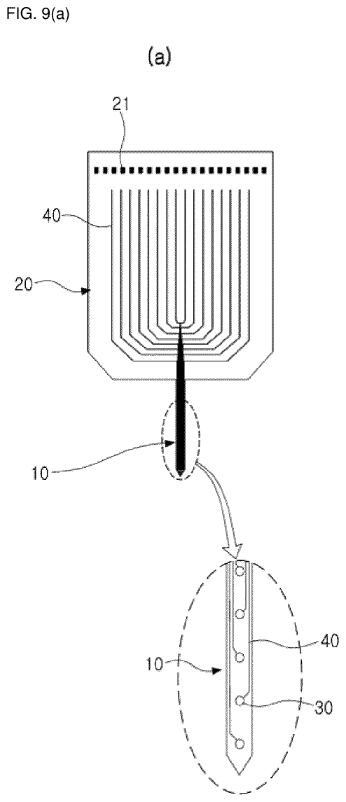

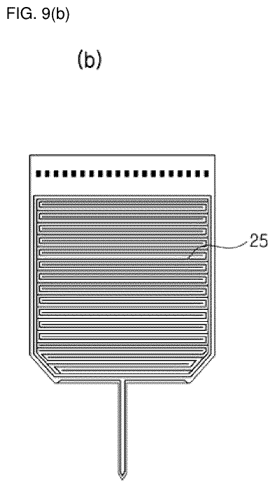

Meanwhile, FIG. 9 shows a schematic diagram of a neural probe according to an embodiment of the present invention. FIG. 9 (a) is a neural probe viewed from one side where electrodes are formed, and FIG. 9 (b) is a schematic diagram of the neural probe viewed from the side opposite to the one side.

The neural probe 1 includes an electrode part 10 and a connection part 20, electrodes 30 (see FIG. 4) are formed in the electrode part 10, and a terminal 21 is formed in the connection part 20. The electrodes 30 of the electrode part 10 and the terminal 21 of the connection part 20 are connected by a wiring 40 and a main body connected to the electrodes 30 of the neural probe is wire-bonded to the terminal 21 of the connection part 20, so that the measurement signal of the human body measured by the electrode 30 is transmitted to the main body, and conversely, the stimulation signal of the main body is transmitted to the human body through the electrodes. The connection part 20 and the electrode part 10 may be connected by one base film 11 (see FIG. 4). That is, a part of the one base film 11 has the electrodes 30 and the wiring 40 formed therein, is cut to be easily inserted into the human body and becomes the electrode part 10, and the other part thereof has the wiring 40 and the terminal 21 formed therein and becomes the connection part 20.

In the present invention, the flexible base film 11 may be a flexible base film 11 made of a flexible material, and may not be advantageous in the case of the connection part 20, which is not the electrode part 10 inserted into the human body. That is, flexibility is a characteristic that is in body. conflict with the securement of desirable durability, so that measures are needed to secure durability in exchange for sacrificing flexibility to some extent.

To this end, a dummy pattern 25 may be formed in the connection part 20 to provide rigidity higher than that of the electrode part 10. It is preferable that the dummy pattern 25 be formed in an area of the neural probe 1 where the electrode 30 or the wiring 40 is not formed and is therefore vulnerable in terms of durability. There is no particular limitation on the directionality of the dummy pattern 25, but it is more preferable to determine the directionality by taking into consideration the directionality of the electrodes 30 and the wiring 40 in the neural probe 1. For example, when the electrodes 30 and the wiring 40 have a vertical pattern, the dummy pattern 25 may have a horizontal pattern. In this case, the degree of flexibility may be different for each pattern direction, and thus, a complementary relationship between the directions of the patterns may be established in terms of the securement of durability.

The present invention may control the bending characteristics while maintaining the thickness and also have optimal satisfactory bending characteristics by further forming the dummy pattern 25 that has only a pattern without being electrically connected to the surface of the flexible neural probe.

According to an embodiment of the present invention, the electrodes 30 are formed only on one surface of the neural probe, and the dummy pattern 25 is formed on the opposite surface of the neural probe. However, the dummy pattern 25 may be formed on both surfaces of the neural probe. In the case where the electrodes 30 are formed on both surfaces of the base film 11, the dummy pattern 25 may be formed in an appropriate portion other than the portions where the electrodes 30 and the wiring 40 are formed, for example, along the outer perimeter of the connection part 20.

The dummy pattern 25 is formed by applying the same material as the electrodes 30 or a material different from that of the electrodes 30 onto the base film 11 through depositing/stacking/coating/etching in the same manner as the electrodes 30. In the case of the dummy pattern 25, electricity is not transmitted therethrough, so that the dummy pattern 25 is not related to the operation of the electrodes 30, and thus, the selection of the material may be freely performed. In the case where the dummy pattern 25 is formed on the flexible base film 11 in this manner, the part where the dummy pattern 25 is formed becomes more rigid than the part where it is not formed. The flexible part of the neural probe, i.e., the electrode part 10, may be stably inserted into the human body, the rigid part thereof, i.e., the connection part 20, may be securely fixed after the insertion, and a drive circuit such as a measurement circuit or a stimulation circuit may be mounted on the connection part 20. Furthermore, a problem such as bending may be prevented during manufacturing.

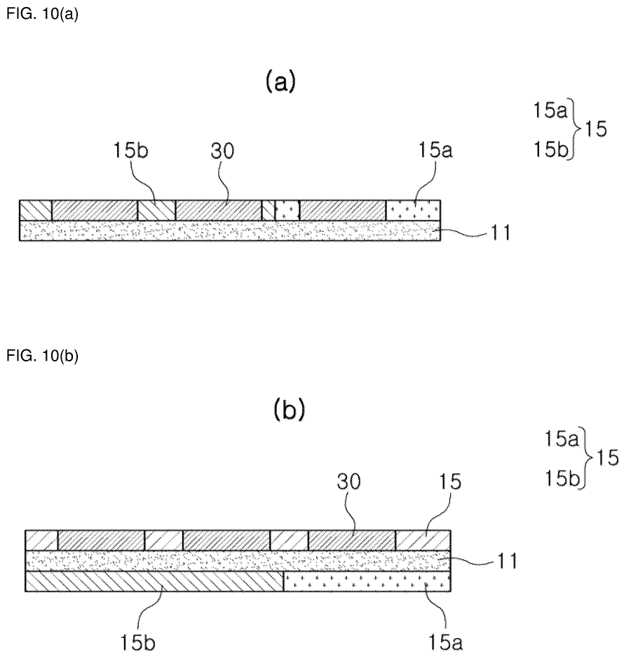

FIG. 10 shows sectional views of the electrode parts 10 of neural probes according to other embodiments of the present invention. FIGS. 10 (a) and 10 (b) show different embodiments.

As shown in the embodiments of FIG. 10, the electrode part 10 includes: a flexible base film 11; electrodes 30 formed on one surface of the base film 11; and an insulation layer 15 covering the base film 11. In the embodiments of FIG. 10, the insulation layer 15 includes a first insulation layer 15a and a second insulation layer 15b that is more flexible than the first insulation layer 15a, and the first insulation layer 15a and the second insulation layer 15b are formed separately according to the location of the base film 11. That is, the second insulation layer 15b is disposed in a portion requiring flexibility, and the first insulation layer 15a is disposed in a portion requiring rigidity and thus supplements the flexible base film 11.

As the portion requiring flexibility, the second insulation layer 15b is disposed on the electrode part 10 that is inserted into the human body, i.e., the opposite side of the portion of the electrode part 10 that is connected to the connection part 20. The first insulation layer 15a is disposed on the portion connected to the connection part 20 and the connection part 20 and thus secures the durability of the neural probe 1.

In FIG. 10 (a), the insulation layer 15 covers the base film 11 while surrounding the electrodes 30. The second insulation layer 15b is disposed on the left side, which is the insertion-side front portion, and the first insulation layer 15a is disposed on the right side, which is the connection part 20. Although the insulation layer 15 is shown as not covering the electrodes 30 in FIG. 10 (a), the first insulation layer 15a or the second insulation layer 15b may be applied even in the case where the insulation layer 15 partially covers the electrodes 30, as shown in FIG. 4.

Meanwhile, in FIG. 10 (b), the insulation layer 15 made of the same material is formed on the surface of the base film 11 where the electrodes 30 are formed, and the insulation layer 15 is formed on the opposite surface of the base film 11 where the electrodes 30 are not formed. The second insulation layer 15b is disposed on a portion requiring flexibility on the opposite surface, and the first insulation layer 15a is disposed on a portion requiring rigidity. In the structure of FIG. 10 (b), the first insulation layer 15a and the second insulation layer 15b are disposed by utilizing one surface of the base film 11 that is not utilized, so that the durability/flexibility of the base film 11 can be secured and it is easy to manufacture the neural probe because the structure is not complicated.

In particular, in the case of the drive circuits, when they are disposed on the flexible base film 11, a problem with durability may occur due to a difference in flexibility with the base film 11. When rigidity is secured through the first insulation layer 15a that is relatively rigid with respect to the base film 11, durability may be secured even when the drive circuits are disposed on the flexible base film 11. Accordingly, it may also be possible to dispose all or a part of the drive circuits on the flexible base film 11. The drive circuits include a measurement circuit and a stimulation circuit, and may also include a storage circuit or a charging circuit.

For example, the flexible base film 11 may include polyimide, polyester, polyphenylene sulfide, etc. The relatively rigid first insulation layer may include epoxy, phenol, etc. The relatively flexible second insulation layer may include liquid polyimide, etc. However, they are not limited thereto, but various materials may be applied to them.

The insulation layer 15 may be applied alone, but may also be applied together with the dummy pattern 25 described above. For example, the dummy pattern 25 may be disposed on the opposite surface of the base film 11, and the relatively rigid first insulation layer 15a covering the dummy pattern 25 and the opposite surface of the base film 11 may be formed.

Furthermore, the insulation layer 15 may be formed not only on the electrode part 10 but also on the connection part 20, and the first insulation layer 15a and the second insulation layer 15b may be separately disposed on one surface where the electrodes 30 are disposed and the opposite surface.

In the present invention, a rigid material is formed in the necessary area of the insulation layer surrounding the flexible electrodes, and a flexible material is applied to other areas, thereby providing a neural probe in a composite structure having both rigidity and flexibility.

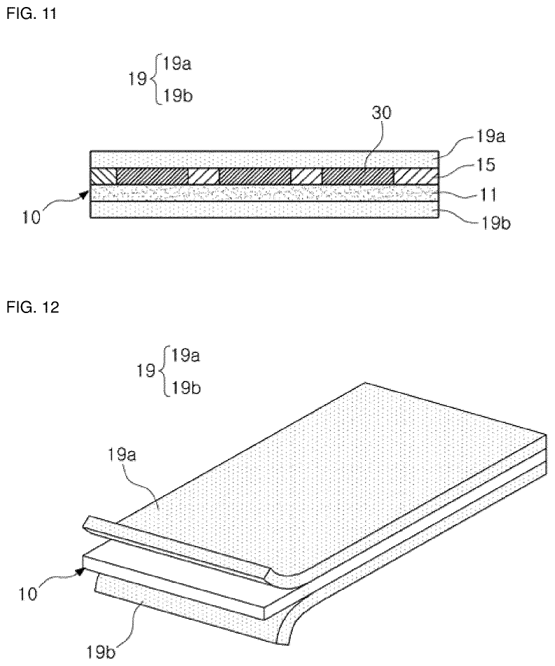

FIG. 11 shows a sectional view of the electrode part of a neural probe according to still another embodiment of the present invention, FIG. 12 shows a perspective view of the embodiment of FIG. 11, and FIG. 13 shows a schematic view of a state in which the embodiment of FIG. 11 is inserted into the human body.

As shown in the embodiment of FIG. 11, the electrode part 10 includes: a flexible base film 11; electrodes 30 formed on one side of the base film 11; an insulation layer 15 covering the base film 11, and protective films 19 disposed on the outermost sides of one and opposite surfaces of the base film 11.

The protective films 19 include a first protective film 19a and a second protective film 19b, the first protective film 19a covers the electrodes 30 and the insulation layer 15, and the second protective film 19b covers the opposite surface of the base film 11. The protective films 19 may each include a film layer, and an adhesive layer that is easily detached from the electrodes 30, the insulation layer 15, and the base film 11.

By using these protective films 19, contamination may be prevented and the surfaces of the electrodes may be physically and chemically protected. In particular, in the case of performing protection in this manner, the movement or handling of a product is made much easier by only simple packaging, so that the packaging materials that have been used in large quantities conventionally can ultimately be simplified and reduced, and various resources required for packaging are also not required.

Furthermore, when the protective films 19 each include the adhesive layer that is easily detached from the electrodes 30, the insulation layer 15, and the base film 11, the neural probe 1 may be inserted into the human body in the state in which the protective films 19 are only partially removed without being completely removed, and then the protective films 19 may be detached naturally as the neural probe 1 is inserted further, as shown in FIG. 13.

The problem in which the electrodes 30 are oxidized over time, and thus, the performance is deteriorated may be overcome by the protective films 19. Furthermore, the present invention enables the insertion of the neural probe into the human body in a simple manner by using a method in which the protective films 19 are attached to the electrode part 10 of the neural probe 1 that is manufactured to be flexible and the adhesive protective films 19 are peeled off at the same time as the neural probe 1 is inserted into the human body.

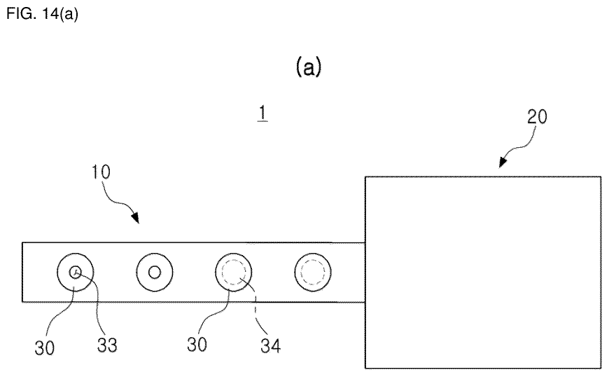

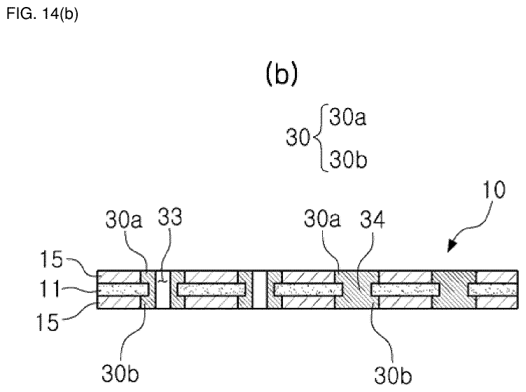

FIG. 14 shows a neural probe 1 according to another embodiment of the present invention.

As shown in FIG. 14, the neural probe 1 includes an electrode part 10, and a connection part 20 connected to the electrode part 10. The electrode part 10 and the connection part 20 are configured such that electrodes 30, a wiring 40, and a terminal 21 (see FIG. 9) are disposed on a flexible base film 11.

In the embodiment of FIG. 14, the electrodes 30 disposed on the base film 11 in the electrode part 10 include first electrodes 30a disposed on one surface of the base film 11 and second electrodes 30b disposed on the other surface thereof. The first and second electrodes 30a and 30b are disposed at corresponding locations with the base film 11 interposed therebetween, and the first and second electrodes 30a and 30b at the corresponding locations are connected through a via or through hole 33 or 34. However, the connection between the first and second electrodes 30a and 30b is not limited thereto.

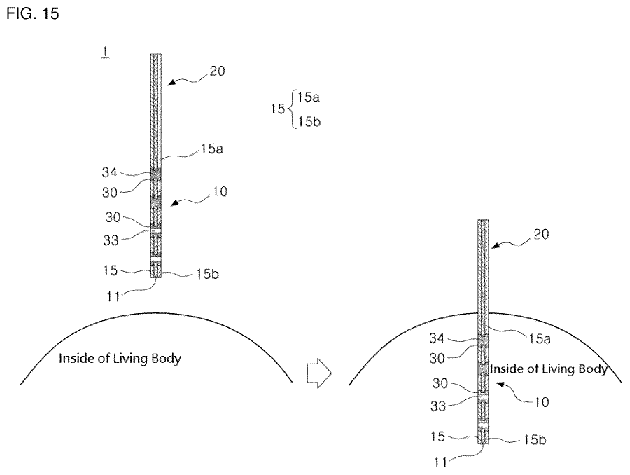

FIG. 15 shows a state in which the electrode part 10 of the neural probe 1 of FIG. 14 is inserted into the human body.

As shown in the present embodiment, the electrodes 30 are formed on both surfaces of the electrode part 10 of the neural probe, and then the electrodes 30 are electrically connected to lower the impedance, thereby improving the precision of bio-signal measurement and stimulation.

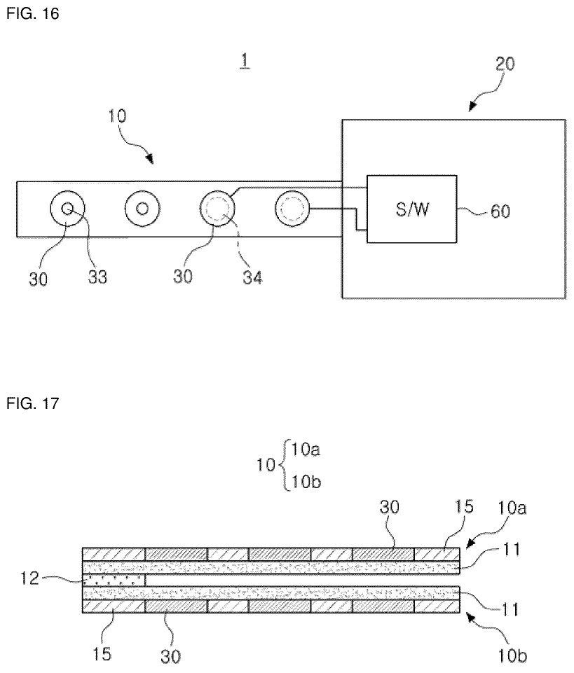

FIG. 16 shows a schematic diagram of another embodiment of a neural probe 1. As shown in FIG. 16, the neural probe 1 includes an electrode part 10 and a connection part 20, and the electrode part 10 includes electrodes 30.

In the embodiment of FIG. 16, the electrodes 30 are electrodes 30 formed on both surfaces, as shown in FIG. 14. However, the electrodes 30 are not limited thereto, but may be electrodes 30 formed on one surface or electrodes 30 formed on both surfaces but not connected through a via hole 33 or a through hole 34 between the electrodes 30. A switching element 60 is disposed in the connection part 20, and the electrodes 30 are connected to the switching element 60. Depending on the connection method between the switching element 60 and the electrodes 30, a parallel structure between the electrodes may be formed, and the impedance of the electrodes may be lowered by the formation of the parallel structure, thereby providing advantages in which the precision of bio-signal measurement is improved and the wide intensity range of bio-stimulation is secured. The parallel structure between the electrodes may be applied to the connection with a drive circuit as well as connection with the switching element 60.

When two types of electrodes 30 are respectively positioned on one and other surfaces of the base film 11 and are not connected to each other, switching elements 60 are also positioned on one and other surfaces of the base film of the connection part 20, and the electrodes 30 positioned on each of the surfaces are connected to the switching element 60 positioned on the same surface, so that the electrodes 30 on each surface operate separately.

FIG. 17 shows a sectional view of the electrode part 10 of a neural probe 1 of another embodiment of the present invention.

As shown in FIG. 17, in the present embodiment, the electrode part 10 includes a plurality of electrode parts 10a and 10b. In the electrode parts 10a and 10b, base films 11 are disposed in parallel with each other, electrodes 30 are formed on the surfaces of the base films 11 that do not face each other, and insulation layers 15 covering the base films 11 while surrounding the electrodes 30 are formed. The individual electrode parts 10a and 10b are connected to each other by a spacer layer 12. That is, the spacer layer 12 is disposed between the surfaces of the electrode parts 10a and 10b facing each other, and the electrode parts 10a and 10b may be disposed apart from each other by the spacer layer 12.

Accordingly, the plurality of electrode parts 10a and 10b having a two-dimensional structure are stacked on top of each other, thereby forming a three-dimensional electrode structure. The spacer layer 12 is included to ensure space between the electrode parts 10a and 10b. The present embodiment may simply put the electrode part 10 having a three-dimensional structure into practice.

Furthermore, the present invention forms the three-dimensional electrode part 10 by stacking base films 11, so that there are tendencies in which the thickness increases and damage to cells increases when the neural probe is inserted into a living body. However, in order to prevent these problems, the spacer layer 12 is interposed to form the gap between the base films 11, so that damage to cells can be minimized.

In this case, the spacer layer 12 may be disposed over the overall area of the electrode part 10, but it may also be disposed only in a partial area. In the case of being disposed in a partial area, it is preferable that the spacer layer 12 be disposed on the end of the electrode part 10 connected to the connection part 20, i.e., on the side of the electrode part 10 opposite to the end of the electrode part 10 inserted into the human body. When the spacer layer 12 is disposed only on one side as described above, there are advantages in that it is easy to secure the connection strength with the connection part 20 and it is also easy to secure the gap between the electrode parts 10.

Although a structure in which the two electrode parts 10a and 10b are stacked is shown in the present embodiment, it may also be possible to stack three or more electrode parts. In the case of an electrode part 10 located in the middle, the human body is inserted into the space secured by the spacer layer 12 to the enable the measurement of signals and the stimulation of the human body.

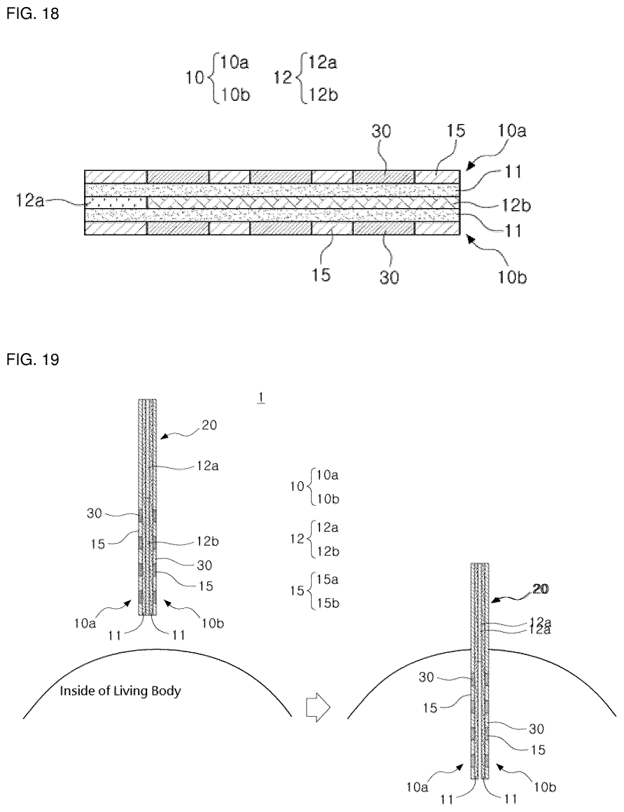

Meanwhile, FIG. 18 shows a sectional view of the electrode part of a neural probe of still another embodiment of the present invention.

As shown in FIG. 18, the electrode part 10 includes a plurality of electrode parts 10a and 10b, and the individual electrode parts 10a and 10b are configured such that base films 11 are disposed in parallel with each other and electrodes 30 are formed on the surfaces of the base films 11 that do not face each other. Furthermore, insulation layers 15 covering the base films 11 while surrounding the electrodes 30 are further included. The individual electrode parts 10a and 10b are connected by a spacer layer 12. That is, the spacer layer 12 is disposed over the overall area between the facing surfaces of the electrode parts 10a and 10b, and the electrode parts 10a and 10b are connected by the spacer layer 12.

The spacer layer 12 includes a first spacer portion 12a made of a bio-insoluble adhesive and a second spacer portion 12b made of a bio-soluble adhesive, and the first spacer portion 12a and the second spacer portion 12b are disposed in each spacer layer, with the second spacer portion 12b being disposed on the end side inserted into the human body.

In the case of the embodiment of FIG. 18, there is the spacer layer 12, but the distance between the electrode parts 10a and 10b is short, so that there is a concern that the ends of the electrode parts 10a and 10b may stick to each other. In the case of insertion in a stuck state, the human body may not be inserted between the electrode parts 10a and 10b, so that there is a concern that the electrodes 30 of the electrode parts 10a and 10b located therebetween may not come into contact with the human body. Accordingly, in the embodiment of FIG. 17, a spacer layer 12 is disposed over the overall area of the electrode parts 10 and a second spacer portion 12b made of a bio-soluble adhesive is disposed to a predetermined distance from the end of the insertion side, so that it is dissolved after being inserted into the human body, allowing body tissues to enter the space between the electrode parts 10a and 10b.

FIG. 19 shows a schematic diagram of a state in which the parts of electrode part 10 connected by a spacer layer 12 are inserted into the human body.

The electrode part 10 of a neural probe 1 includes a plurality of electrode parts 10a and 10b, and the individual electrode parts 10a and 10b are configured such that base films 11 are disposed in parallel with each other and electrodes 30 are formed on the surfaces of the base films 11 that do not face each other. Furthermore, insulation layers 15 covering the base films 11 while surrounding the electrodes 30 are further included. The individual electrode parts 10a and 10b are separated by the spacer layer 12. That is, the spacer layer 12 is disposed over the overall area between the facing surfaces of the electrode parts 10a and 10b, and the electrode parts 10a and 10b are connected by the spacer layer 12.

The electrode parts 10a and 10b and the connection part 20 are formed on connected base films 11, and the connection part 20 is also connected by the spacer layer 12. Although a bio-insoluble adhesive may be used for the spacer layer 12 of the connection portion 20, other adhesives, such as even bio-soluble adhesives, may be used because the connection part 20 is not a part to be inserted into the human body.

The spacer layer 12 includes a first spacer portion 12a made of a bio-insoluble adhesive and a second spacer portion 12b made of a bio-soluble adhesive, and the first spacer portion 12a and the second spacer portion 12b are disposed in the spacer layer 12, with the first spacer portion 12a being disposed only on the connection part (20)-side end of the electrode part 10 and the second spacer portion 12b being disposed in most of the area of the electrode part 10.

Meanwhile, the insulation layer 15 includes a first insulation layer 15a and a second insulation layer 15b that is more flexible than the first insulation layer 15a, the first insulation layer 15a may be disposed at a location of the electrode part 10 close to the connection part 20, and the second insulation layer 15b may be disposed at the insertion end. That is, the second insulation layer 15b is disposed at the insertion end requiring flexibility, and the first insulation layer 15a is disposed at the portion connected to the connection part 20, thereby supplementing the flexible base films 11.

In the embodiment of FIG. 19, before insertion, the electrode parts 10a and 10b are maintained in an integrated state, so that the size of the electrode part 10 is minimized, and thus, damage to tissues such as nerves and cells is minimized when the electrode part 10 is inserted into a living body. After insertion, the first spacer portion 12a, which is a bio-soluble adhesive, is dissolved, so that a gap is formed between the electrode parts 10a and 10b, making it easy to insert the electrode part 10 into a living body. Accordingly, more electrodes may be disposed in a three-dimensional array, so that diagnosis and treatment can be performed more effectively.

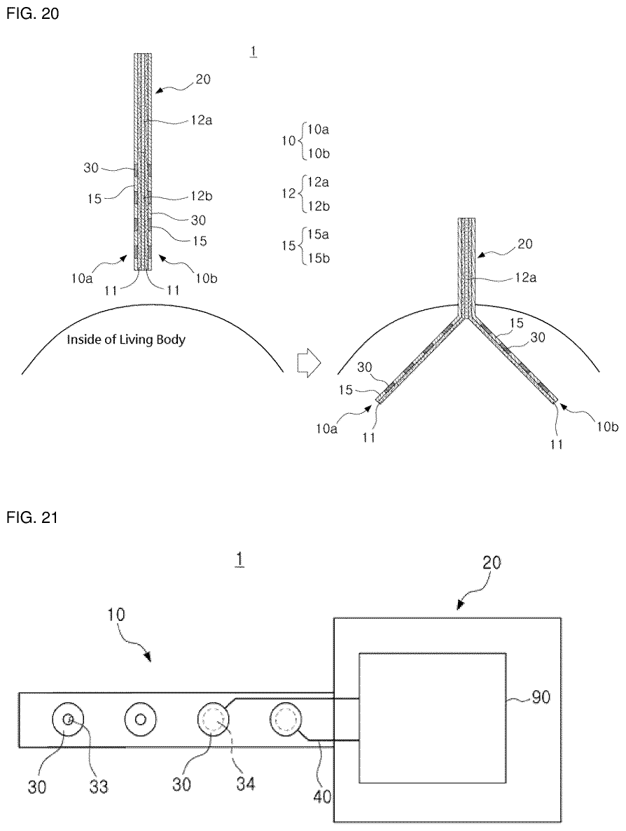

In this case, as shown in FIG. 20, each of electrode parts 10a and 10b may be configured to include a material layer that varies in the degree of deformation when the temperature changes so that the electrode parts 10a and 10b can be opened inside the human body. After being inserted into the body, the second spacer portions 12b are dissolved, and the electrode parts 10a and 10b are deformed by body temperature, and accordingly, the electrode parts 10a and 10b can be opened.

The materials of the electrodes 30 applied to individual unit electrode parts 10 are the same, but the materials of the base films 11 may be different. Since the individual materials may have different coefficients of thermal expansion, the degrees of deformation thereof are different, so that there is a high possibility that they will be separated from each other.

In addition Furthermore, the unit electrode parts 10 are made of a shape memory material so that they maintain a minimum size before insertion into the human body, thereby reducing the invasive range. After insertion into the human body, the gap between the unit electrode probes is changed in a direction that increases, so that bio-signal measurement or bio-stimulation can be performed in a wider range.

Furthermore, in the present invention, the spacer is made of a bio-soluble material so that it is dissolved after insertion into a living body, thereby minimizing damage to cells, increasing the contact area between the electrode probe and cells, and also improving the ease of contact.

FIG. 21 is a schematic diagram of a neural probe according to still another embodiment of the present invention.

In the case of FIG. 21, an electrode part 10 and a connection part 20 are configured by disposing electrodes 30 or a drive circuit 90 on the same base film 11, and the electrodes 30 of the electrode part 10 are connected to the drive circuit 90 through a wiring 40. The electrodes 30 are formed on both surfaces, and the electrodes 30 on both surfaces are connected through a via hole 33 or a through hole 34. Although not shown in the drawing, a dummy pattern 25 (see FIG. 9) is formed on the connection part 20, and a relatively rigid first insulation layer 15a (see FIG. 10) is formed on the connection part 20.

In the present invention, the drive circuit is installed in the neural probe and electrically connected to the neural probe, so that a connection structure for the drive circuit that was previously installed outside the neural probe and connected by a wire is omitted, thereby simplifying the structure of a system including the neural probe, reducing the weight of the system, and also ensuring the ease of handling of the system. Furthermore, a neural probe drive circuit such as a neural signal recording device may be mounted in a rigid area, so that a separate control unit is not required, thereby reducing the overall size of the device.

Although the present invention has been described in more detail above with reference to the embodiments, the present invention is not necessarily limited to these embodiments, but various modifications may be made within a scope that does not depart from the technical spirit of the present invention. Therefore, the embodiments disclosed in the present invention are not intended to limit the technical spirit of the present invention but are intended to describe it, and the scope of the technical spirit of the present invention is not limited by these embodiments. The scope of protection of the present invention should be interpreted based on the attached claims, and all technical spirits within the equivalent scope should be interpreted as being included in the scope of the rights of the present invention.

1. A neural probe comprising a plurality of electrode parts to be inserted into a human body;
wherein the plurality of electrode parts each comprise a flexible substrate, electrodes disposed on one surface of the flexible substrate, and an insulation layer covering the one surface of the flexible substrate; and
wherein the plurality of electrode parts are stacked.

2. The neural probe of claim 1, wherein a spacer layer is disposed on a side opposite to an end to be inserted into the human body between the plurality of electrode parts.

3. The neural probe of claim 1, wherein a spacer layer is disposed between the plurality of electrode parts, at least the spacer layer comprises a first spacer portion made of a bio-insoluble adhesive and a second spacer portion made of a bio-soluble adhesive, and the first and second spacer portions are disposed in the spacer layer with the second spacer portion disposed on an end side to be inserted into the human body.

4. The neural probe of claim 1, wherein the insulation layer comprises a first insulation layer, and a second insulation layer having higher flexibility than the first insulation layer.

5. The neural probe of claim 4, wherein part of the insulation layer covering the flexible substrate is the second insulation layer.

6. The neural probe of claim 5, further comprising a connection part connected to the electrode parts and provided with a terminal that is electrically connected to electrodes;
wherein an insulation layer covering the connection part and an insulation layer covering a portion of the electrode part connected to the connection part are the first insulation layer.

7. The neural probe of claim 6, wherein a drive circuit connected to the electrodes is mounted at a location corresponding to the first insulation layer.

8. The neural probe of claim 7, further comprising a wiring connecting the electrodes and the drive circuit and formed on the substrate;
wherein the wiring connects at least some of the electrodes in parallel to the drive circuit.

9. The neural probe of claim 1, wherein the electrode part and the connection part comprise an additional insulation layer covering the substrate on an opposite surface of the substrate.

10. The neural probe of claim 9, wherein the additional insulation layer comprises a first additional insulation layer, and a second additional insulation layer having higher flexibility than the first additional insulation layer.

11. The neural probe of claim 10, wherein an additional insulation layer covering the connection part and an additional insulation layer covering a portion of the electrode part connected to the connection part are the first additional insulation layer.

12. The neural probe of claim 11, wherein a drive circuit connected to the electrodes is mounted at a location corresponding to the first insulation layer.

13. The neural probe of any one of claims 1 to 12, wherein the insulation layer covers some of the electrodes and comprises through holes so that the electrodes have exposed surfaces.

14. The neural probe of any one of claims 1 to 12, further comprising a protective film covering at least one surface of the electrode part.

15. The neural probe of any one of claims 1 to 12, wherein the electrodes comprise a first electrode disposed on one surface of the substrate and a second electrode disposed on an opposite surface thereof.

16. The neural probe of claim 15, wherein the first electrode disposed on the one surface of the substrate and the second electrode disposed on the opposite surface thereof are disposed at locations corresponding to each other.

17. The neural probe of claim 16, further comprising a via or through hole formed through the substrate to connect the first and second electrodes.

18. A neural probe comprising an electrode part to be inserted into a human body, wherein:
the electrode part comprises:
a flexible substrate;
electrodes disposed on one surface of the flexible substrate; and
an insulation layer covering the flexible substrate on the one surface; and
the electrodes comprise a first electrode disposed on one surface of the substrate and a second electrode disposed on an opposite surface thereof.

19. The neural probe of claim 18, wherein the first electrode disposed on the one surface of the substrate and the second electrode disposed on the opposite surface thereof are disposed at locations corresponding to each other.

20. The neural probe of claim 19, further comprising a via or through hole formed through the substrate to connect the first and second electrodes.

21. The neural probe of any one of claims 18 to 20, further comprising a protective film covering at least one surface of the electrode part.
